(19) United States
(12) Patent Application Publication
(10) Pub. No.: US 2026/0290570 A1
(43) Pub. Date: Sep. 24, 2026

(54) SURGICAL DATA DASHBOARD

(71) Applicants: Digital Surgery Limited, London (GB); Covidien LP, Mansfield, MA (US)

(72) Inventors: Nilay Mukherjee, North Andover, MA (US); Arvind K. Ramadorai, Lexington, MA (US); Abhinav Chintakunta, New Hyde Park, NY (US); Hyun Kiy Song, London (GB); Carole RJ Addis, London (GB); George Bruce Murgatroyd, Hitchin (GB); Stefano Passaretti, London (GB)

(21) Appl. No.: 19/473,658

(22) PCT Filed: Apr. 12, 2024
(86) PCT No.: PCT/EP2024/060090

§ 371 Date: Oct. 8, 2025

Related U.S. Application Data
(60) Provisional application No. 63/459,390, filed on Apr. 14, 2023.

(51) Int. Cl. G16H 40/63; A61B 34/00

(52) U.S. Cl. CPC G16H 40/63; A61B 34/25

(57) ABSTRACT

Techniques are described for mapping data recorded during one or more surgical procedures to a user-interactive dashboard. The mapping includes processing the data using various machine learning and other resource-intensive computations. The mapping further includes creating user-interactive elements that facilitate navigating the results of the analysis.

### BACKGROUND

The present invention relates in general to computing technology and relates more particularly to computing technology for generation and display of a surgical data dashboard.

A building block in the effort to standardize surgery is the existence of a set of metrics, which can meaningfully capture surgical variability. Furthermore, identifying key surgical approaches within a set of cases paves the way to correlating techniques with patient outcomes and efficiency. Identifying and displaying such information can be resource intensive.

### SUMMARY

According to one or more aspects, a computer-implemented method for surgical data dashboard generation and display can include analyzing a surgical dataset including information associated with a plurality of surgical procedures performed and determining one or more metrics that quantify a plurality of aspects of the surgical procedures. The computer-implemented method can also include generating a surgical data dashboard that summarizes the aspects of the surgical procedures based on the one or more metrics including a workflow analysis with a filter control. The computer-implemented method can further include adjusting one or more visualizations of the surgical data dashboard based on a user selection through the filter control.

According to one or more aspects, a system includes a memory system and a processing system coupled to the memory system and configured to execute instructions to perform a plurality of operations. The operations can include analyzing a surgical dataset including information associated with a plurality of surgical procedures performed, where the surgical dataset can include two or more of: surgical tool data, surgical video data, and/or configuration information. The operations can also include determining one or more metrics that quantify a plurality of aspects of the surgical procedures, where at least one of the one or more metrics is determined based on at least one machine learning model. The operations can further include generating a surgical data dashboard that summarizes the aspects of the surgical procedures based on the one or more metrics including a workflow variation view configured to display a workflow standardization and a variation over surgery. One or more visualizations of the surgical data dashboard can be adjusted based on a user selection.

According to one or more aspects, a computer program product includes a memory device with computer-readable instructions stored thereon, where executing the computer-readable instructions by one or more processing units causes the one or more processing units to perform a plurality of operations. The operations can include analyzing a surgical dataset including information associated with a plurality of surgical procedures performed, where the surgical dataset includes surgical tool data, surgical video data, and configuration information. The operations can also include determining one or more metrics that quantify a plurality of aspects of the surgical procedures and generating a surgical data dashboard that summarizes the aspects of the surgical procedures based on the one or more metrics including a case summary view that summarizes the surgical procedures as a plurality of cases and includes search interface to perform text-based searches to filter displayed content of the case summary view. The operations can further include adjusting one or more visualizations of the surgical data dashboard based on a user selection.

### BRIEF DESCRIPTION OF THE DRAWINGS

The specifics of the exclusive rights described herein are particularly pointed out and distinctly claimed in the claims at the conclusion of the specification. The foregoing and other features and advantages of the aspects of the invention are apparent from the following detailed description taken in conjunction with the accompanying drawings in which:

FIG. 1 depicts a surgical data dashboard according to one or more aspects;

FIG. 2 depicts a workflow analysis view of a surgical data dashboard according to one or more aspects;

FIG. 3 depicts a workflow variation view of a surgical data dashboard according to one or more aspects;

FIG. 4 depicts a robotic analysis view of a surgical data dashboard according to one or more aspects;

FIG. 5 depicts a reachability metric view of a surgical data dashboard according to one or more aspects;

FIG. 6 depicts a performance view of a surgical data dashboard according to one or more aspects;

FIG. 7 depicts an error tracking view of a surgical data dashboard according to one or more aspects;

FIG. 8 depicts an instrument analysis view of a surgical data dashboard according to one or more aspects;

FIG. 9 depicts a case summary view of a surgical data dashboard according to one or more aspects;

FIG. 10 depicts a case view of a surgical data dashboard according to one or more aspects;

FIG. 11 depicts a system for analyzing data according to one or more aspects;

FIG. 12 depicts a flowchart of a method of training models for insight generation according to one or more aspects; and

FIG. 13 depicts a block diagram of a computer system according to one or more aspects described herein.

The diagrams depicted herein are illustrative. There can be many variations to the diagrams and/or the operations described herein without departing from the spirit of the invention. For instance, the actions can be performed in a differing order, or actions can be added, deleted, or modified. Also, the term “coupled” and variations thereof describe having a communications path between two elements and do not imply a direct connection between the elements with no intervening elements/connections between them. All of these variations are considered a part of the specification.

### DETAILED DESCRIPTION

Exemplary aspects of the technical solutions described herein include systems and methods for generation, display, and interaction with a surgical data dashboard. To objectively provide feedback to surgical service providers, such as hospital departments, medical practices, surgeons, etc., technical solutions described herein provide a methodology to quantify surgical procedures, including quantifying one or more steps, actions, and phases of a surgical procedure. The resulting quantification can be used for standardization based on the evaluation of several similarity metrics. Further, pattern recognition techniques can be used to identify unique approaches associated with each surgical service provider. Technical solutions described herein facilitate the use of qualitative measures of surgical performance to standardize variable surgical practices to improve patient safety and surgical outcomes and lower costs, as well as efficient utilization of system resources.

Further yet, the technical solutions described herein facilitate quantifying training or gaps in the training of a trainee surgeon. Further, the technical solutions described herein can facilitate identifying protocols and/or steps to use for particular surgical procedures to improve patient outcomes and efficiency. The technical solutions can also facilitate streamlining surgical procedures so that hospitals and other service providers can be ready for different cases that can be predicted.

An input dataset is analyzed using machine learning models in one or more examples of the technical solutions described herein. The input dataset includes surgical data of multiple surgical procedures performed. The multiple surgical procedures can be of the same type, e.g., Laparoscopic Roux-en-Y Gastric Bypass. Alternatively, or in addition, the surgical procedures can be of several different types, e.g., gastric bypass, cataract surgery, knee arthroscopy, etc. The surgical procedures are performed at different institutions (e.g., hospitals) and by different surgeons. The surgical data includes video data, e.g., endoscopic video, for each surgical procedure. In addition, the surgical data can include surgical instrumentation data, e.g., instrument activations, energy supplied, etc., during the surgical procedure. The dataset can include additional data for each surgical procedure, such as hospital identification, surgeon identification, patient body sensor measurements during the surgical procedure, etc. The surgical data can include automatically identified phases, steps, actions performed during the surgical procedure using machine learning models.

The machine learning models used by the technical solutions herein can include artificial neural networks, such as deep neural networks, convolutional neural networks, recurrent neural networks, encoders, decoders, or other such machine learning models.

In one or more examples, each case in an input dataset was segmented into surgical phases, for example, based on Touch Surgery™ standardized annotation guidelines. For example, a dataset can include several cases (hundreds or even thousands) of a surgical procedure, such as a Laparoscopic Roux-en-Y Gastric Bypass, cataract surgery, etc., obtained from a distinct surgical service provider.

The technical solutions described herein facilitate computing at least the following metrics including parameters/features according to Table 1.

TABLE 1
Computed Metrics

| Parameter/Feature | Calculation Details |
|---|---|
| Case Duration | Time between “Patient In” annotation and “Patient Out” |
| | annotation. |
| Patient In to Cut | Time between “Patient In” annotation and start of video |
| | recording/“Operation Start” annotation. |
| Operating Time/Cut to Close | Time between start of video recording/“Operation Start” |
| | annotation and “Operation End” annotation. |
| Close to Patient Out | Time between “Operation End” annotation and “Patient |
| | Out” annotation. |
| Camera Out % | Summation of Camera Out periods divided by the |
| | Operating Time × 100. |
| Efficiency and Safety | Benchmarks for “Component” and “Proficient” defined |
| Performance Benchmarking | using Performance metric distributions of clinical data |
| | collected. |
| Maximum Error Level | Defined using Error Tracking metric distributions of |
| | clinical data collected. |
| Proficiency Measure | Number of Safety Performance metrics user has reached |
| (Safety) % | the “Proficient” benchmark for divided by the total |
| | number of metrics × 100. |
| Proficiency Measure | Number of Efficiency Performance metrics user has |
| (Efficiency) % | reached the “Proficient” benchmark for divided by the |
| | total number of metrics × 100. |
| Case Duration Outlier | Outlier detection analysis calculation for case durations |
| | using a Boxplot Measure suitable for skewed |
| | distributions. |
| Phase Duration Outlier | Outlier detection analysis calculation for phase durations |
| | using a Boxplot measure suitable for skewed distributions. |
| Instrument Standardization | A measure between 0 (instruments used different in every |
| | case) and 1 (instruments used are identical in every case). |
| Unused Pref Card Instruments | Number of instruments unused in case but included in |
| | Preference Card (represents potential cost wastage - |
| | instruments opened/requiring sterilization). |
| Used Pref Card Instruments | Number of instruments used in case and included in |
| | Preference Card. |
| Used Instruments not | Number of instruments used in case but not included in |
| on Pref. Card | Preference Card (represents potential time wastage - |
| | instruments not present in operating theatre but required). |
| Workflow Standardization | A measure between 0 (phase workflow different in every |
| | case) and 1 (phase workflow the same in every case). |
| Variation over Surgery | A measure of variation (entropy) across a set of aligned |
| | surgical workflows. |
| Expected Case Values | Expected case value ranges (e.g. case duration) can be |
| | predicted using factors to identify similar cases (e.g., |
| | patient factors). |
| Significant Workflow Deviation | Indicates if a case workflow significantly deviates from a |
| Indicator (Workflow Variation | surgeon's usual workflow. |
| tag) | |
| Significant Instrument Deviation | Indicates if a case instrument list significantly deviates |
| Indicator (Instrument Variation | from a surgeon's usual instrument list. |
| tag) | |
| Patient Outlier | Indicates if a patient's characteristics significant deviate |
| | from the usual patient characteristics. Can use |
| | multivariate outlier detection algorithm. |
| Case Complexity/Patient Factor | Method to remove the effect of case factors to allow for |
| Effect Elimination | comparable data analytics such as Propensity Score |
| | Matching, Stratification, Regression Adjustment etc. |
| Learning Curve Prediction | Method to estimate a confidence interval for learning |
| | curve prediction. |
| Duration System Time On (s) - | Duration between the event when the Start Surgery button |
| Learning Curve | is pressed on ORTI Display and the event when the |
| | System Power button is used to deactivate system either |
| | on Tower or armrest of Surgeon Console. |
| Duration First Trocar Attach to | Duration between the event when the first trocar is |
| Last Trocar Detach (s) - Learning | attached to the robot arm and the event when the last |
| Curve | trocar is detached from the robot arm (and no instruments |
| | are attached). |
| Duration Active Console Time (s) - | Total duration when either of two Surgeon Hand |
| Learning Curve | Controllers (AKA Control Devices) are in unlocked |
| | mode. |
| Duration when surgeon is | Total duration when 1) the surgeon is inattentive (i.e., |
| inattentive and a minimum of one | head tracking system determines that surgeon is not |
| Surgeon Hand Controller (AKA | looking at the console) AND 2) at least one Surgeon hand |
| control device) is in unlocked | controller is unlocked. The case where the surgeon is |
| mode (s) - Novice Error | Inattentive and both Surgeon Hand Controllers are locked |
| | does not count. |
| Number of Instrument | A continuous curvilinear movement of the First Proximal |
| translational movements - | Joint (FPJ) of the instrument of 3 mm or more counts as a |
| Learning Curve | movement. Changing the movement direction by 90 |
| | degrees for a continuous curvilinear movement of at least |
| | 3 mm, counts as a new movement. This will be measured |
| | for each instrument that is in use. Instrument wristed |
| | motion and jaw open/close motions are not considered as |
| | instrument movement. |
| Instrument translational path | Total distance travelled by instrument (Based on FPJ |
| length (mm) - Learning Curve | movement that is tracked when surgeon is attentive and at |
| | least one Surgeon Hand Controller is unlocked. This does |
| | NOT include instrument wrist or jaw movement). This |
| | will be measured for each instrument that is in use. |
| Instrument translational velocity | The velocity with which the FPJ of the instrument moves. |
| (mm/s) - Learning Curve | This will be measured for each instrument that is in use. |
| Primary Instrument idle time | Duration of time when primary instrument is idle and |
| while unlocked and attentive (s) - | when system is in a mode where surgeon is looking at the |
| Novice Error | monitor, at least one Surgeon hand controller is unlocked, |
| | and the surgeon is not moving the instruments. |
| | Instrument is defined to be idle if it is not in movement. |
| | This will be measured for each instrument that is in use. |
| Number of Curvilinear Camera | Movement is defined as curvilinear movement of the tip |
| movements - Learning Curve | of the endoscope. A continuous curvilinear movement of |
| | 3 mm or more counts as a movement. Changing the |
| | movement direction by 90 degrees for a continuous |
| | curvilinear movement of at least 3 mm, counts as a new |
| | movement. |
| Number of camera “roll” | The number of times when a continuous roll of endoscope |
| movements - Learning Curve | occurs above a threshold limit (to be defined). Roll is |
| | distinct from curvilinear movement. |
| Camera path length (mm/s) - | Total curvilinear distance travelled by tip of endoscope |
| Learning Curve | when surgeon is looking at the monitor and at least one |
| | surgeon hand controller is unlocked. This will be |
| | measured for each endoscope that is used. |
| Clutch usage during position | Total number of occurrences when any clutch is engaged |
| control - Learning Curve | (foot pedal or left hand trigger or right hand trigger on |
| | Surgeon Hand Controller) while system is in position |
| | control (Surgeon looking at the monitor and at least one |
| | surgeon hand controller is unlocked). This does not |
| | include attentive-inattentive transitions from head |
| | tracking. |
| Fourth Arm Swap Failure Rate | Number of events when the fourth arm is successfully |
| (Novice Error) | swapped using either screen based GUI or foot pedal |
| | divided by the number of events when the fourth arm is |
| | attempted to be swapped using either screen based GUI or |
| | foot pedal. |
| Energy Bank Activation Failure | Number of events when energy bank was successfully |
| Rate (Novice Error) | activated divided by the number of events when energy |
| | bank was attempted to be activated - this includes all |
| | energy activations (monopolar-cut, coag; bipolar; |
| | ligasure- cut, seal). |
| Energy Activation Failure Rate | Number of events when energy was successfully activated |
| (Novice Error) | divided by the number of events when energy was |
| | attempted to be activated - this includes all energy |
| | activations (monopolar-cut, coag; bipolar; ligasure- cut, |
| | seal). |
| Number of calibrated Instrument - | Number of detachments of a calibrated Instrument |
| instrument exchanges - | followed by an attachment of a instrument with a different |
| Learning Curve | serial number to instrument drive unit (IDU), for every |
| | arm that is in use. |
| Number of endoscope-endoscope | Number of events a different endoscope is plugged-in. |
| exchanges - Learning Curve | |
| Number of endoscope-instrument | Number of detachments of an endoscope followed by an |
| exchanges - Learning Curve | attachment of a instrument or vice versa to IDU, for every |
| | arm that is in use. |
| Hand controller workspace range | The larger of the two radii of motion of the user's |
| (cm) - Learning Curve | working volume on left and right hand Surgeon Hand |
| | Controllers. |
| Workspace limit of Robot arm/ | Number of times workspace limit reached on robot arm/ |
| instrument - Novice Error | instruments. |
| WorkSpace Limit of Surgeon | Number of times workspace limit reached on either or |
| Hand controllers - Novice Error | both Surgeon Hand Controllers. |

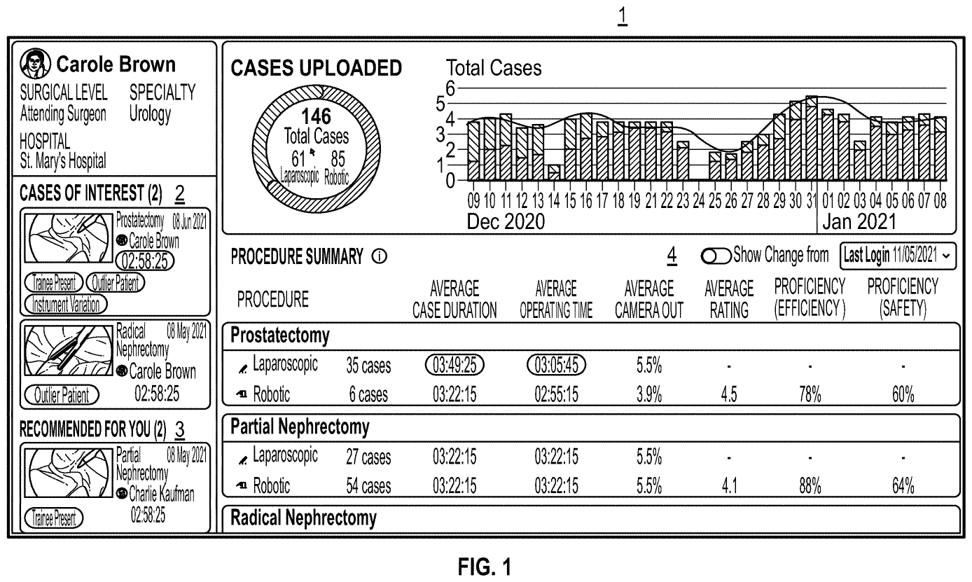

Computed metrics can be displayed on a surgical data dashboard 1 as depicted in the example of FIG. 1. The surgical data dashboard 1 can be surgeon-specific, department-specific, hospital-specific, or provide details across several combinations. A surgeon-specific dashboard is shown in the example of FIG. 1. The surgical data dashboard 1 can be configured to show information such as total surgical videos analyzed for the surgeon, different types of surgical procedures performed, duration of each procedure, duration of the camera being inside the patient, and one or more of the above metrics computed for each of the surgical procedures. In some examples, one or more metrics can be aggregated in the displayed information. The displayed information can be filtered and/or interacted with in some examples. A cases of interest interface 2 can be displayed as a graphical summary that is selectable including, for example, a case name, a surgeon, a duration, a thumbnail image, and one or more tags. A recommended for you interface 3 can be displayed as a graphical summary that is selectable including, for example, a case name, a surgeon, a duration, a thumbnail image, and one or more tags. The surgeons may vary between the cases of interest interface 2 and the recommended for you interface 3. A change control 4 can be selectable to filter a view of the surgical data dashboard 1 with respect to dates. The dates can be selectable through a pulldown, for instance, that may correspond to a login history of the current user. This can allow for a customized time history view associated with the login history of the user to select data groupings which have been updated with respect to a viewing history of the user. The surgical data sources can include a combination of surgical tool data, surgical video data, and metrics derived from the surgical tool data (e.g., instruments, robotic system data, etc.), the surgical video data, and/or configuration information.

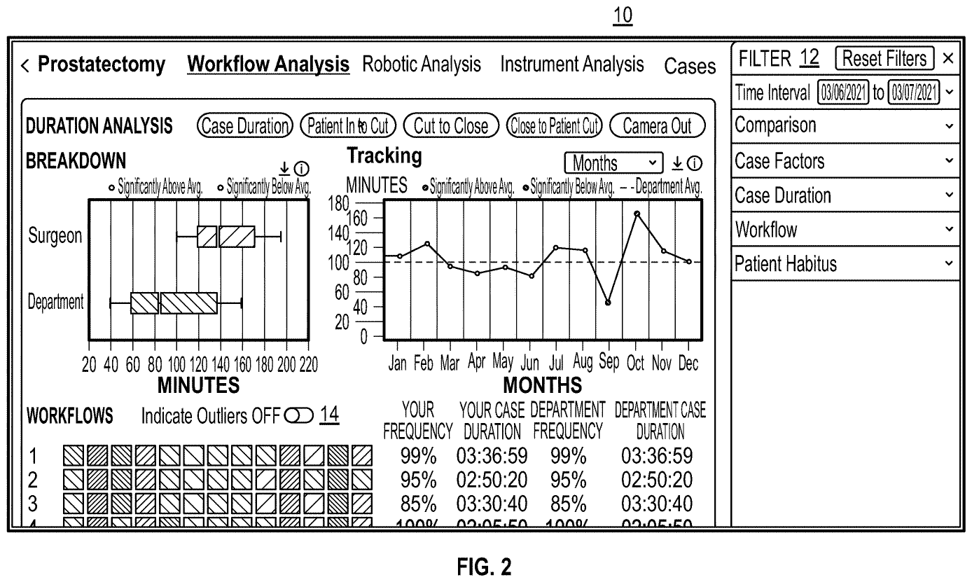

In some examples, a user can interact with the surgical data dashboard 1 to display further details. For example, analysis of a particular type of surgical procedure can be performed as shown in an example dashboard image as a workflow analysis view 10 in FIG. 2. The user can interact with the workflow analysis view 10 to compare a surgical workflow and other metrics associated with a surgical procedure performed by a first surgeon with the same surgical procedure performed by other surgeons or by the same first surgeon at other times, for example. The user can interact with the elements shown in FIG. 2 to playback segments of videos from the other surgical procedures in some examples. In some examples, the user can use a filter control 12 to filter the surgical procedures to be compared, such as procedures during a particular duration, with specific case factors, with particular patient traits, with particular workflow characteristics, specific case durations, etc. Other controls can include an indicate outliers control 14 that can be selected to show workflows with outliers identified or not identified.

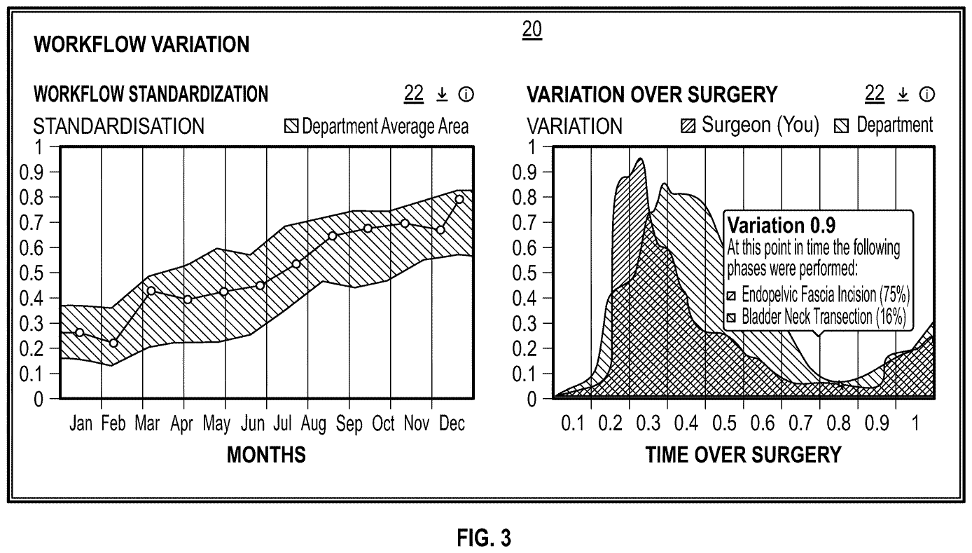

In some aspects, the surgical data dashboard 1 can display a comparison of the various selected surgical procedures using visualizations. For example, a visualization can indicate different workflows adopted for each of the surgical procedures. A workflow can be depicted with each phase/action being represented by a color-coded block, for example. Further, timings for each phase can be displayed with a comparison between a particular surgeon/group and others. The comparison metrics can be aggregated to compute a workflow standardization score and/or a variation score, as depicted in a workflow variation view 20 of FIG. 3. Scores can be plotted over time to provide the user a visual depiction of the performance of the surgical procedure by a particular surgeon/group of surgeons. The workflow variation view 20 can also include download controls 22 to trigger a download of selected data and/or images (e.g., plots and/or underlying data).

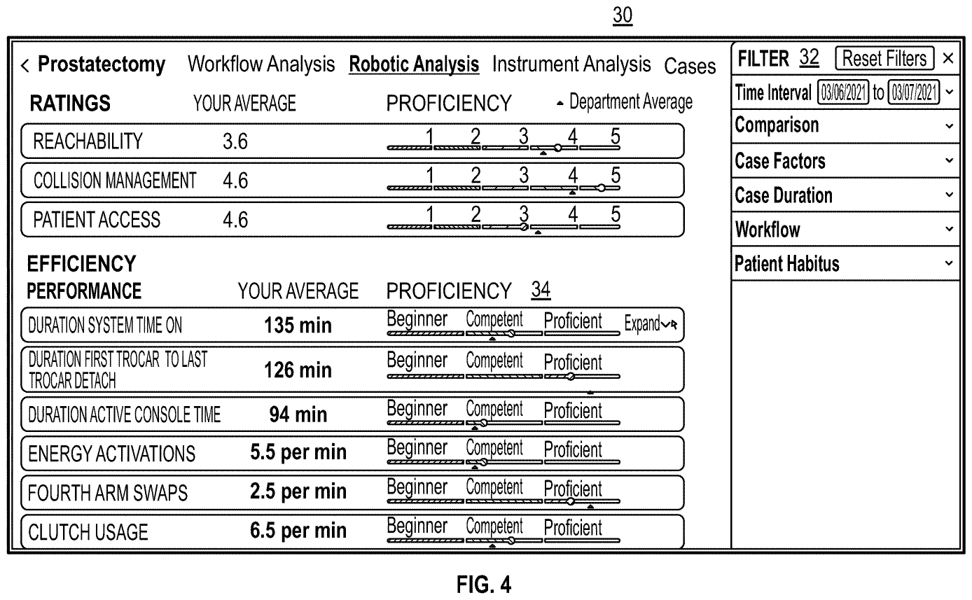

Further yet, the surgical data dashboard 1 can facilitate displaying the metrics and performing an analysis of the use of the computer-assisted surgery system. For example, the surgical data dashboard 1 can include a robotic analysis view 30 of FIG. 4, which can display data, such as ratings and performance efficiency with user averages and proficiency relative to department data, that can include scores from other users. In some examples, the user can use a filter control 32 to filter the surgical procedures to be compared, such as procedures during a particular duration, with specific case factors, with particular patient traits, with particular workflow characteristics, specific case durations, etc. Other controls can include an expand control 34 that can be selected to change the level of detail depicted through the robotic analysis view 30.

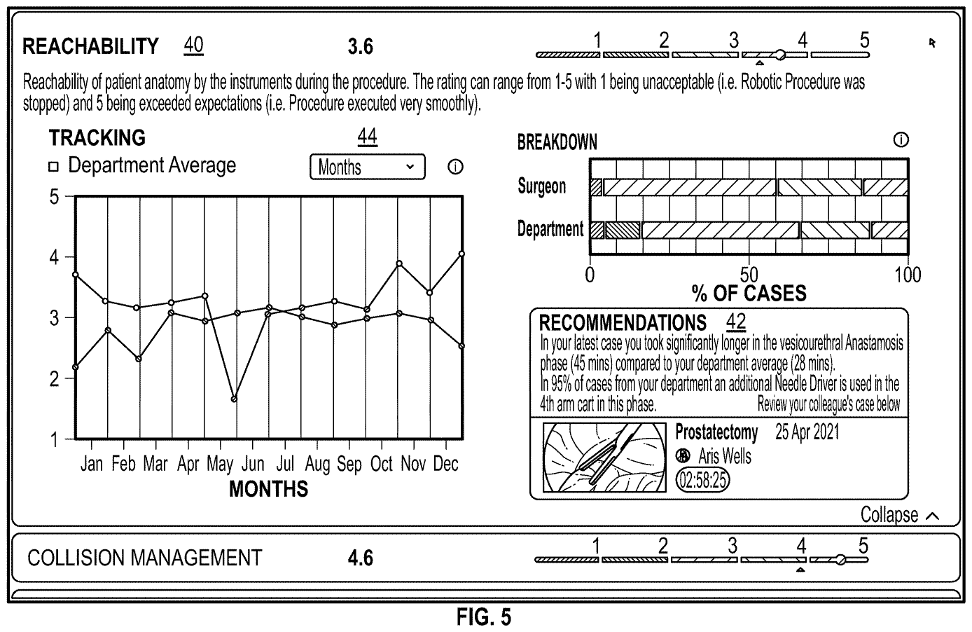

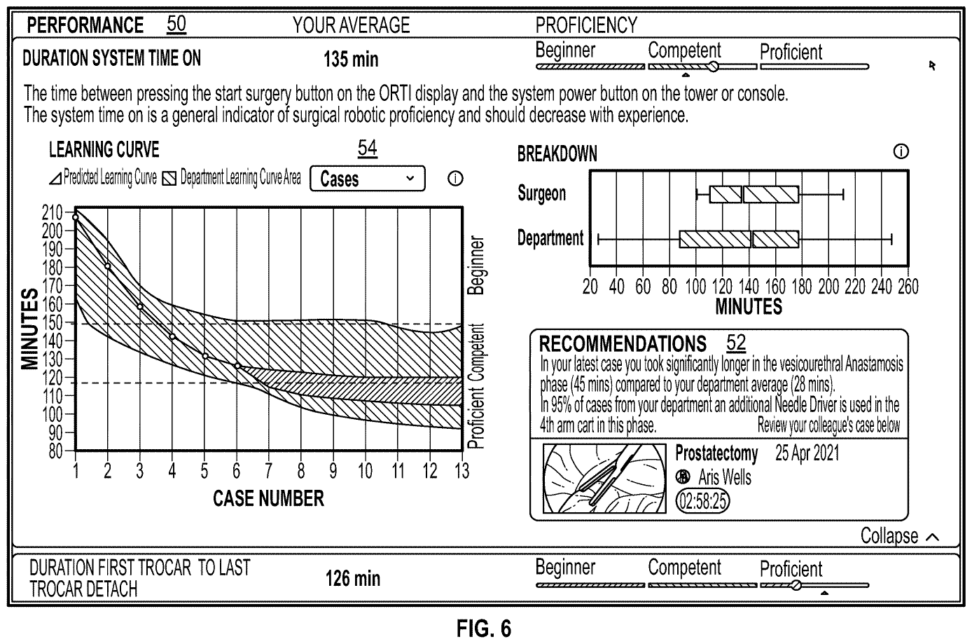

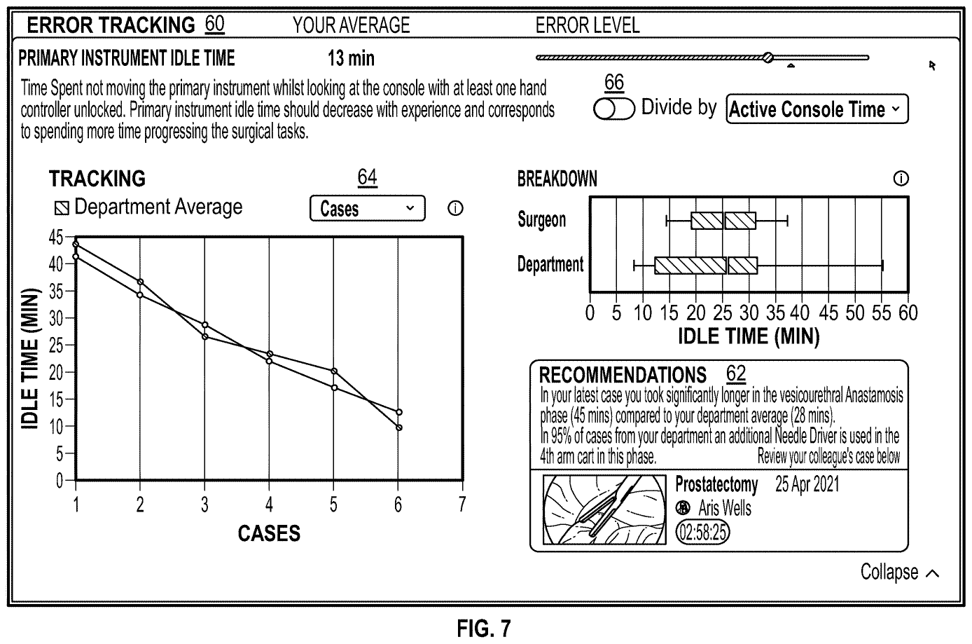

One or more of the metric views can be interacted with to see further details, such as a reachability metric view 40 of the surgical data dashboard 1 in FIG. 5 and a performance view 50 of the surgical data dashboard 1 in FIG. 6. Further, based on the metrics, the surgical data dashboard 1 can display recommendations to improve the surgical actions/phase in future surgical procedures, such as recommendations 42 of FIG. 5 and recommendations 52 of FIG. 6 identifying potential performance improvements. Various controls can adjust aspects of the reachability metric view 40 and performance view 50, such as a time base selector 44 to set a time base for tracking data display and/or a learning curve selector 54 (e.g., cases) to group data for proficiency ratings. The surgical data dashboard 1 also can display the duration of use/idle-time of one or more surgical instruments used in the surgical procedure, for instance, as part of an error tracking view 60 of the surgical data dashboard 1 of FIG. 7. Based on the usage (or idle time), several metrics and comparisons can be computed and displayed. Recommendations 62 can also be made to improve operational efficiency, for example. Other recommendations can also be made. Other controls can include a tracking control 64 (e.g., cases) to select a data source for tracking plotting and a division control 66 (e.g., an active console time) to define how data should be broken down for summarizing and display.

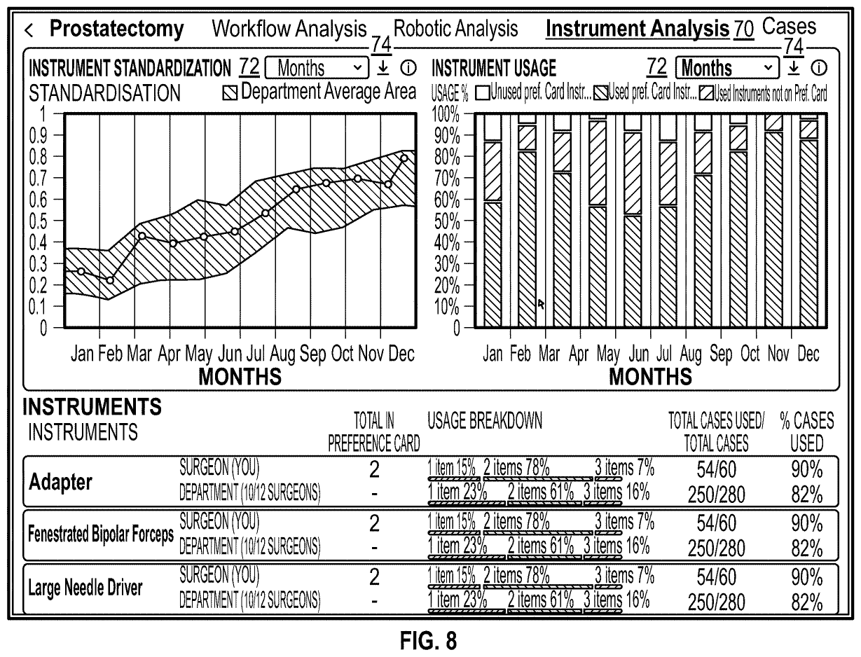

In addition, the usage of specific surgical instruments can be analyzed, and user-interactive visualizations based on the analysis can be displayed to the user via an instrument analysis view 70 of the surgical data dashboard 1 of FIG. 8. The usage metrics of the instruments can be categorized based on several parameters. For example, a surgeon can request one or more instruments specific instruments for a surgical procedure in one or more examples. The usage of the instruments identifies the actual usage of the specific instruments requested and those that were actually used but not requested. The surgeon can update his/her preferences accordingly. Additionally, the surgeon can visualize instruments used by other surgeons during their surgical procedures of the same type and alter his/her procedures accordingly. The usage metrics of an instrument can be standardized so that comparison across different surgical procedures, across different surgeons, across different hospitals, etc., can be performed. Display controls can include, for example, a time base control 72 to establish a time interval for plotting data and a download control 74 to download images and/or underlying data associated with various plots.

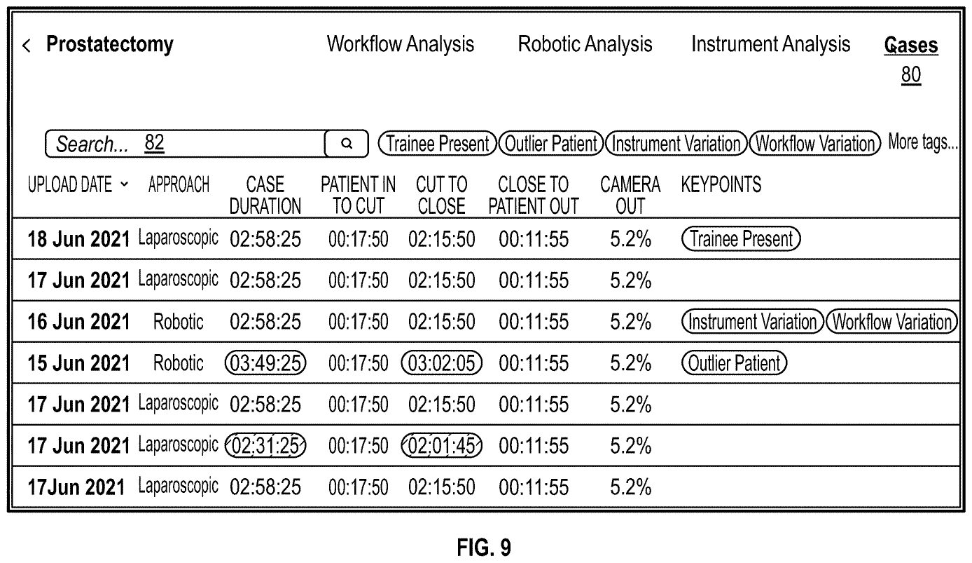

Additionally, metrics can be visualized on a per surgical procedure basis using a case summary view 80 of the surgical data dashboard 1 of FIG. 9. A record of a surgical procedure can be further embellished with one or more tags to identify particular characteristics, events, etc. For example, the tags can include trainee present, outlier patient, instrument variation, workflow variation, etc. In one or more examples, the tags can be automatically added by analyzing the surgical data associated with the surgical procedures. The surgical data can include video, audio, patient EMR, etc. The user can filter the displayed surgical procedures. The user can also interact with the surgical procedures. For example, the user can select a surgical procedure to view the workflow used, and other metrics of that surgical procedure. The user can further select and playback the video, audio, or particular segments of the video and audio associated with that surgical procedure. Other controls can include a search interface 82 to perform text-based searches to filter the contents of the case summary view 80.

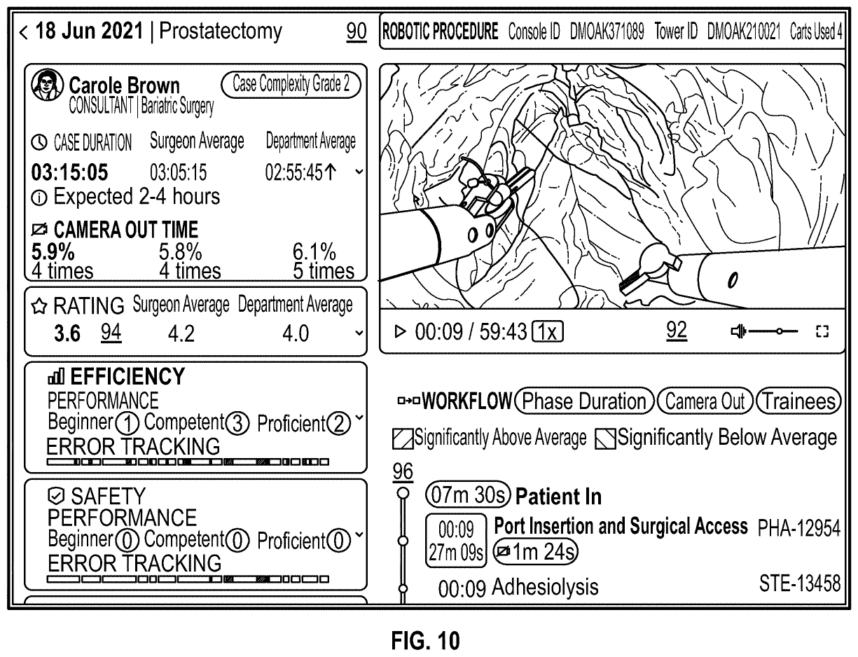

The user can further view one or more events associated with the surgical procedure (e.g., using tags) and interact with the representations of such characteristics to directly playback video/audio associated with that characteristic using a case view 90 of the surgical data dashboard 1 of FIG. 10. The case view 90 may be reached through a selection from the case summary view 80 of FIG. 9. The case view 90 can include a video interface 92 to play video of a selected case. A user can rate the surgical procedure through a rating interface 94 and add comments and/or annotations through the case view 90. Particular characteristics or events during the surgical procedure can be marked along a timeline 96 of the surgical procedure. The characteristics or events can include instrument related events, e.g., unavailability, technical issue, etc. The characteristics or events can also include surgical events, e.g., bleeding, particular surgical actions, completion of a phase, etc. The characteristics or events can also identify portions of the surgical events performed by a surgeon, a trainee, a particular person, a robot, etc. In one or more examples, the timeline 96 can be interactive. Interacting with a particular characteristic or event causes the video playback to change and show portions relevant to that particular characteristic or event. The characteristics and events are identified automatically using machine learning in one or more examples.

The visual attributes of one or more user interface elements in the dashboard(s) can be based on one or more metrics.

Technical solutions herein facilitate computing and using metrics to identify surgically meaningful approaches from a surgical dataset with granular annotations. The decomposition of the dataset into approaches and presentation of the characteristic features of each approach agreed with the medical teams' understanding of the procedure facilitates using the techniques herein to automatically provide insights into the advantages and disadvantages of different approaches. The technical solutions described herein can facilitate identifying characteristics of the approaches that provide a strong basis for automatic classification of a workflow. Further, the technical solutions described herein can facilitate identifying standardization, its correlation with durations (of surgical procedures/phases), and surgical outcomes. The technical solutions herein further facilitate comparing different approaches used by different surgeons, hospitals, etc.

Technical solutions described herein facilitate using machine learning analysis to a surgical dataset of multiple surgical procedures, with annotations at a coarse level (surgical phase/objective). Further, the dataset analyzed by the technical solutions described herein uses surgical procedures performed by several surgeons, several institutions (e.g., hospitals). The technical solutions described herein provide automatic identification of surgical approaches across multiple institutions, multiple surgeons, etc. Further, the technical solutions herein provide analysis using the standardization metrics.

Technical solutions described herein can facilitate measurable improvement in surgical interventions by objectively quantifying surgical standardization, process efficiency, and patient outcomes. A robust standardization metric computed herein facilitates adherence to an approach to be measured and changes over time as a result of training to be tracked and quantified.

The technical solutions herein facilitate comparing different surgical procedures, surgeons, surgery providers based on one or more of the above metrics. In one or more examples, correlation(s) between one or more of the above metrics and patient outcomes, surgery efficiency, and other such results can be computed. Additional advantages and effects of the technical solutions can be identified by persons skilled in the art.

Exemplary aspects of technical solutions described herein relate to, among other things, devices, systems, methods, computer-readable media, techniques, and methodologies for using machine learning and computer vision to automatically predict regions of interest in surgical data and generate modified visualizations. More generally, aspects can include detection, tracking, and predictions associated with one or more structures, the structures being deemed to be critical for an actor involved in performing one or more actions during a surgical procedure (e.g., by a surgeon).

In some instances, a computer-assisted surgical (CAS) system is provided that uses one or more machine-learning models, trained with surgical data, to augment environmental data directly sensed by an actor involved in performing one or more actions during a surgical procedure (e.g., a surgeon). Such augmentation of perception and action can increase action precision, optimize ergonomics, improve action efficacy, enhance patient safety, and improve the standard of the surgical process. The output of the one or more machine-learning models can also be an alert used to trigger a real-time notification of a deviation in the surgical procedure or highlight a region outside of a current region of interest, for example.

The surgical data provided to train the machine-learning models can include data captured during a surgical procedure, as well as simulated data. The surgical data can include time-varying image data (e.g., a simulated/real video stream from different types of cameras) corresponding to a surgical environment. The surgical data can also include other types of data streams, such as audio, radio frequency identifier (RFID), text, robotic sensors, other signals, etc. The machine-learning models are trained to predict and identify, in the surgical data, “structures” including particular tools, anatomic objects, actions being performed in the simulated/real surgical stages. In one or more aspects, the machine-learning models are trained to define one or more parameters of the models so as to learn how to transform new input data (that the models are not trained on) to identify one or more structures. During the training, the models receive as input, one or more data streams that may be augmented with data indicating the structures in the data streams, such as indicated by metadata and/or image-segmentation data associated with the input data. The data used during training can also include temporal sequences of one or more input data.

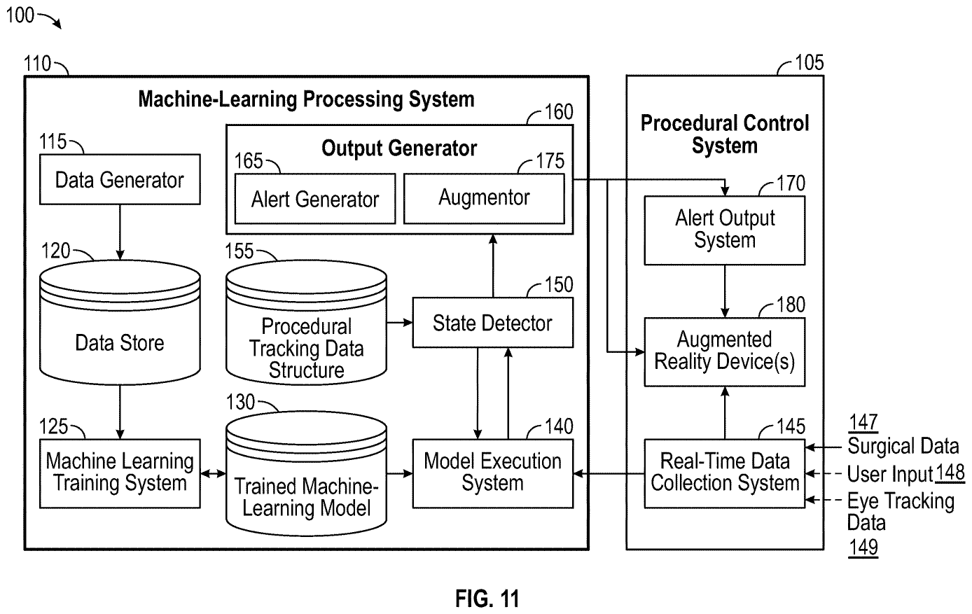

FIG. 11 shows a system 100 for predicting surgical phases and structures in surgical data and generating adaptive and interactive visualizations using machine learning according to one or more aspects. System 100 uses data streams in the surgical data to identify procedural states according to some aspects. System 100 includes a procedural control system 105 that collects image data and coordinates outputs responsive to predicted structures and states. The procedural control system 105 can include one or more devices (e.g., one or more user devices and/or servers) located within and/or associated with a surgical operating room and/or control center. System 100 further includes a machine-learning processing system 110 that processes the surgical data using one or more machine-learning models to identify a procedural state (also referred to as a phase or a stage), which is used to identify a corresponding output. It will be appreciated that machine-learning processing system 110 can include one or more devices (e.g., one or more servers), each of which can be configured to include part or all of one or more of the depicted components of the machine-learning processing system 110. In some instances, a part, or all of machine-learning processing system 110 is in the cloud and/or remote from an operating room and/or physical location corresponding to a part, or all of procedural control system 105. For example, the machine-learning training system 125 can be a separate device, (e.g., server) that stores its output as the one or more trained machine-learning models 130, which are accessible by the model execution system 140, separate from the machine-learning training system 125. In other words, in some aspects, devices that “train” the models are separate from devices that “infer,” i.e., perform real-time processing of surgical data using the trained models 130.

Machine-learning processing system 110 includes a data generator 115 configured to generate simulated surgical data, such as a set of virtual images, or record surgical data from ongoing procedures, to train one or more machine-learning models. Data generator 115 can access (read/write) a data store 120 with recorded data, including multiple images and/or multiple videos. The images and/or videos can include images and/or videos collected during one or more procedures (e.g., one or more surgical procedures). For example, the images and/or video may have been collected by a user device worn by a participant (e.g., surgeon, surgical nurse, anesthesiologist, etc.) during the surgery, and/or by a non-wearable imaging device located within an operating room.

Each of the images and/or videos included in the recorded data can be defined as a base image and can be associated with other data that characterizes an associated procedure and/or rendering specifications. For example, the other data can identify a type of procedure, a location of a procedure, one or more people involved in performing the procedure, surgical objectives, and/or an outcome of the procedure. Alternatively, or in addition, the other data can indicate a stage of the procedure with which the image or video corresponds, rendering specification with which the image or video corresponds and/or a type of imaging device that captured the image or video (e.g., and/or, if the device is a wearable device, a role of a particular person wearing the device, etc.). Further, the other data can include image-segmentation data that identifies and/or characterizes one or more objects (e.g., tools, anatomical objects, etc.) that are depicted in the image or video. The characterization can indicate the position, orientation, or pose of the object in the image. For example, the characterization can indicate a set of pixels that correspond to the object and/or a state of the object resulting from a past or current user handling.

Data generator 115 identifies one or more sets of rendering specifications for the set of virtual images. An identification is made as to which rendering specifications are to be specifically fixed and/or varied. Alternatively, or in addition, the rendering specifications that are to be fixed (or varied) are predefined. The identification can be made based on, for example, input from a client device, a distribution of one or more rendering specifications across the base images and/or videos, and/or a distribution of one or more rendering specifications across other image data. For example, if a particular specification is substantially constant across a sizable data set, the data generator 115 defines a fixed corresponding value for the specification. As another example, if rendering-specification values from at least a predetermined amount of data span across a range, the data generator 115 can define the rendering specifications based on the range (e.g., to span the range or to span another range that is mathematically related to the range of distribution of the values).

A set of rendering specifications can be defined to include discrete or continuous (finely quantized) values. A set of rendering specifications can be defined by a distribution, such that specific values are to be selected by sampling from the distribution using random or biased processes.

One or more sets of rendering specifications can be defined independently or in a relational manner. For example, if the data generator 115 identifies five values for a first rendering specification and four values for a second rendering specification, the one or more sets of rendering specifications can be defined to include twenty combinations of the rendering specifications or fewer (e.g., if one of the second rendering specifications is only to be used in combination with an incomplete subset of the first rendering specification values or the converse). In some instances, different rendering specifications can be identified for different procedural phases and/or other metadata parameters (e.g., procedural types, procedural locations, etc.).

Using the rendering specifications and base image data, the data generator 115 generates simulated surgical data (e.g., a set of virtual images), which is stored at the data store 120. For example, a three-dimensional model of an environment and/or one or more objects can be generated using the base image data. Virtual image data can be generated using the model to determine—given a set of particular rendering specifications (e.g., background lighting intensity, perspective, zoom, etc.) and other procedure-associated metadata (e.g., a type of procedure, a procedural state, a type of imaging device, etc.). The generation can include, for example, performing one or more transformations, translations, and/or zoom operations. The generation can further include adjusting the overall intensity of pixel values and/or transforming RGB values to achieve particular color-specific specifications.

A machine-learning training system 125 uses the recorded data in the data store 120, which can include the simulated surgical data (e.g., set of virtual images) and actual surgical data to train one or more machine-learning models. The machine-learning models can be defined based on a type of model and a set of hyperparameters (e.g., defined based on input from a client device). The machine-learning models can be configured based on a set of parameters that can be dynamically defined based on (e.g., continuous or repeated) training (i.e., learning, parameter tuning). Machine-learning training system 125 can use one or more optimization algorithms to define the set of parameters to minimize or maximize one or more loss functions. The set of (learned) parameters can be stored at a trained machine-learning model data structure 130, which can also include one or more non-learnable variables (e.g., hyperparameters and/or model definitions).

A model execution system 140 can access the machine-learning model data structure 130 and accordingly configure one or more machine-learning models for inference (i.e., prediction). The one or more machine-learning models can include, for example, a fully convolutional network adaptation, an adversarial network model, or other types of models as indicated in data structure 130. The one or more machine-learning models can be configured in accordance with one or more hyperparameters and the set of learned parameters.

The one or more machine-learning models, during execution, can receive, as input, surgical data 147 to be processed and generate one or more inferences according to the training. For example, the surgical data 147 can include data streams (e.g., an array of intensity, depth, and/or RGB values) for a single image or for each of a set of frames representing a temporal window of fixed or variable length in a video. The surgical data 147 that is input can be received from a real-time data collection system 145, which can include one or more devices located within an operating room and/or streaming live imaging data collected during the performance of a procedure. The surgical data 147 can include additional data streams, such as audio data, RFID data, textual data, measurements from one or more surgical instruments/sensors, etc., that can represent stimuli/procedural state from the operating room. In some aspects, the real-time data collection system 145 can also receive eye tracking data 149 as a supplemental input, for example, based on one or more optical sensors that track the eyes of a surgeon performing a surgical procedure. The various inputs from different devices/sensors are synchronized before being input to the model.

In some aspects, the real-time data collection system 145 can receive user input 148 from one or more devices, such as a tablet computer, a tactile input, or other such device. For example, the user input 148 can allow a surgical team to draw a contour of interest for tracking and enhancing over time. Structures identified through user-based identification via user input 148 can be tracked in combination with or in place of structures identified through machine learning. Further, the user input 148 may support modifying contours or other features identified for enhancement by machine learning. Records of the user input 148 can be tracked to assist in further training of machine learning and/or can be tracked as preferences available for use by the surgical team in future surgical procedures.

The one or more machine-learning models can analyze the surgical data 147, and in one or more aspects, predict and/or characterize structures included in the visual data from the surgical data 147. The visual data can include image and/or video data in the surgical data 147. The prediction and/or characterization of the structures can include segmenting the visual data or predicting the localization of the structures with a probabilistic heatmap. In some instances, the one or more machine-learning models include or are associated with a preprocessing or augmentation (e.g., intensity normalization, resizing, cropping, etc.) that is performed prior to segmenting the visual data. An output of the one or more machine-learning models can include image-segmentation or probabilistic heatmap data that indicates which (if any) of a defined set of structures are predicted within the visual data, a location and/or position and/or pose of the structure(s) within the image data, and/or state of the structure(s) associated with a proposed region of interest. The location can be a set of coordinates in the image data. For example, the coordinates can provide a bounding box that defines the proposed region of interest. Alternatively, the coordinates provide boundaries that surround the structure(s) being predicted as the proposed region of interest. The one or more machine-learning models can be trained to perform higher-level predictions and tracking, such as predicting a phase of a surgical procedure and tracking one or more surgical instruments used in the surgical procedure. In some aspects, the machine-learning models can synthesize an image adjustment or provide supporting data for another component to synthesize an image adjustment.

A state detector 150 can use the output from the execution of the machine-learning model to identify a state within a surgical procedure (“procedure”). A procedural tracking data structure can identify a set of potential states that can correspond to part of a performance of a specific type of procedure. Different procedural data structures (e.g., and different machine-learning-model parameters and/or hyperparameters) may be associated with different types of procedures. The data structure can include a set of nodes, with each node corresponding to a potential state. The data structure can include directional connections between nodes that indicate (via the direction) an expected order during which the states will be encountered throughout an iteration of the procedure. The data structure may include one or more branching nodes that feed to multiple next nodes and/or can include one or more points of divergence and/or convergence between the nodes. In some instances, a procedural state indicates a procedural action (e.g., surgical action) that is being performed or has been performed and/or indicates a combination of actions that have been performed. In some instances, a procedural state relates to a biological state of a patient undergoing a surgical procedure. For example, the biological state can indicate a complication (e.g., blood clots, clogged arteries/veins, etc.), pre-condition (e.g., lesions, polyps, etc.).

Each node within the data structure can identify one or more characteristics of the state. The characteristics can include visual characteristics. In some instances, the node identifies one or more tools that are typically in use or availed for use (e.g., on a tool tray) during the state, one or more roles of people who are typically performing a surgical task, a typical type of movement (e.g., of a hand or tool), etc. Thus, state detector 150 can use the segmented data generated by model execution system 140 that indicates the presence and/or characteristics of particular objects within a field of view to identify an estimated node to which the real image data corresponds. Identification of the node (and/or state) can further be based upon previously detected states for a given procedural iteration and/or other detected input (e.g., verbal audio data that includes person-to-person requests or comments, explicit identifications of a current or past state, information requests, etc.).

In some aspects, state and phase values determined by the state detector 150 can be fed back to be used by one or more machine-learning models executed by the model execution system 140. For example, a machine-learning model can be used to generate features for use by the state detector 150, and state or phase determinations of the state detector 150 can become inputs for other machine-learning models or networks executed by the model execution system 140 as part of a chain or collection of machine-learning models trained to further enhance machine-learning results.

An output generator 160 can use the state to generate an output. Output generator 160 can include an alert generator 165 that generates and/or retrieves information associated with the state and/or potential next events. For example, the information can include details as to warnings and/or advice corresponding to current or anticipated procedural actions. The alert generator 165 can be configured to communicate with one or more other systems, such as procedural control system 105, to provide notice or trigger actions based on the information. The information can further include one or more events to monitor. The information can identify the next recommended action. An alert may include highlighting a structure that is not within the current region of interest, such as highlighting bleeding, anatomical structures, and/or surgical structures where further attention may be needed to shift the focus of the surgeon.

The user feedback can be transmitted to an alert output system 170, which can cause the user feedback to be output via a user device and/or other devices that is (for example) located within the operating room or control center. The user feedback can include a visual, audio, tactile, or haptic output that is indicative of the information. The user feedback can facilitate alerting an operator, for example, a surgeon, or any other user of the system 100.

Output generator 160 can also include an augmentor 175 that generates or retrieves one or more graphics and/or text to be visually presented on (e.g., overlaid on) or near (e.g., presented underneath or adjacent to or on separate screen) real-time capture of a procedure. Augmentor 175 can further identify where the graphics and/or text are to be presented (e.g., within a specified size of a display). In some instances, a defined part of a field of view is designated as being a display portion to include augmented data. In some instances, the position of the graphics and/or text is defined so as not to obscure the view of an important part of an environment for the surgery and/or to overlay particular graphics (e.g., of a tool) with the corresponding real-world representation. A modified visualization of the surgical procedure can include incorporating an image adjustment in a real-time output of the video of the surgical procedure, such as adjusting one or more of contrast, color, and focus in a region of interest. Further examples of adjustments can include full new image synthesis, recoloring, pastelization, and/or other enhancement techniques known in the art. The image adjustments may appear as a virtual light source within the image and can be concentrated with a greater intensity near the centroid of the region of interest, for example.

Augmentor 175 can send the graphics and/or text and/or any positioning information to an augmented reality device 180, which can integrate the graphics and/or text with a user's environment in real-time as an augmented reality visualization. Augmented reality device 180 can include a pair of goggles that can be worn by a person participating in part of the procedure. It will be appreciated that, in some instances, the augmented display can be presented at a non-wearable user device, such as at a computer or tablet. The augmented reality device 180 can present the graphics and/or text at a position as identified by augmentor 175 and/or at a predefined position. Thus, a user can maintain a real-time view of procedural operations and further view pertinent state-related information.

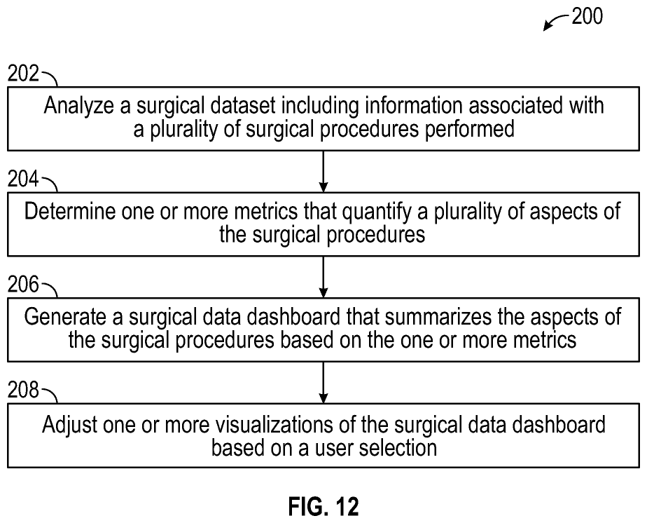

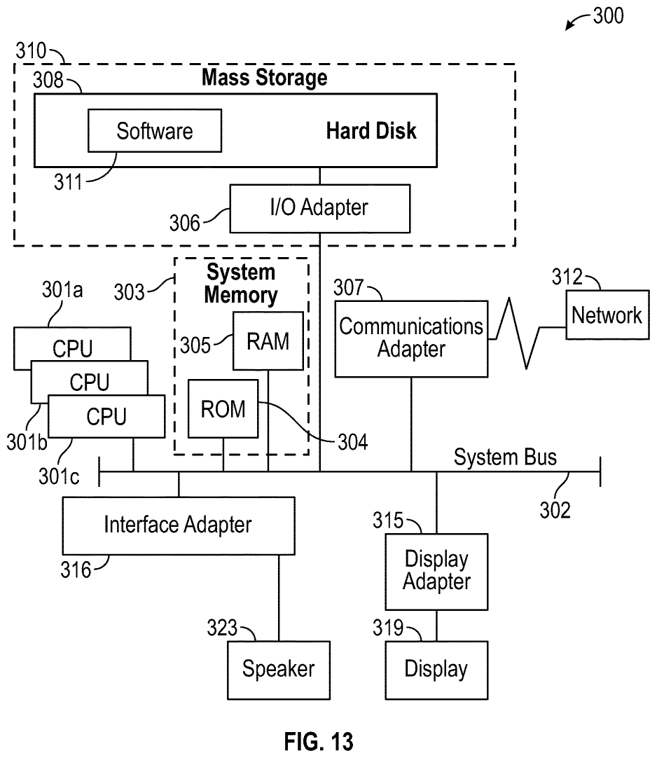

Turning now to FIG. 12, a flowchart of a method 200 for surgical data dashboard generation and display is generally shown in accordance with one or more aspects. All or a portion of method 200 can be implemented by a system that includes one or more computer systems, such as computer system 300 of FIG. 13, such as one or more processors executing instructions stored in memory to provide an interactive user interface.

At block 202, a processor can analyze a surgical dataset including information associated with a plurality of surgical procedures performed. At block 204, the processor can determine one or more metrics that quantify a plurality of aspects of the surgical procedures. At block 206, the processor can generate a surgical data dashboard that summarizes the aspects of the surgical procedures based on the one or more metrics including a workflow analysis with a filter control. At block 208, the processor can adjust one or more visualizations of the surgical data dashboard based on a user selection through the filter control.

According to some aspects, the one or more metrics can be displayed in user interactive elements.

In some aspects, the surgical dataset can include two or more of: surgical tool data, surgical video data, and/or configuration information (e.g., user, time/date, surgical procedure type, etc.).

In some aspects, the one or more metrics can include time based metrics, camera position based metrics, efficiency based metrics, error based metrics, outlier based metrics, workflow based metrics, and/or safety based metrics.

In some aspects, the one or more metrics can include variation based metrics, complexity metrics, predictive metrics, surgical tool movement based metrics, and/or surgeon learning based metrics.

In some aspects, the surgical data dashboard can include a cases of interest interface, a recommended for you interface, and a change control that provides a selectable filter to control data selection in displaying the surgical data dashboard.

In some aspects, the surgical data dashboard can include one or more of: a workflow analysis view that compares a surgical workflow and other metrics associated with a surgical procedure, a workflow variation view, a robotic analysis view, a reachability metric view, a performance view, an error tracking view, an instrument analysis view, and a case summary view.

In some aspects, a system includes a memory system and a processing system coupled to the memory system and configured to execute instructions to perform a plurality of operations. The operations can include analyzing a surgical dataset including information associated with a plurality of surgical procedures performed, where the surgical dataset can include two or more of: surgical tool data, surgical video data, and/or configuration information. The operations can also include determining one or more metrics that quantify a plurality of aspects of the surgical procedures, where at least one of the one or more metrics is determined based on at least one machine learning model. The operations can further include generating a surgical data dashboard that summarizes the aspects of the surgical procedures based on the one or more metrics including a workflow variation view configured to display a workflow standardization and a variation over surgery. One or more visualizations of the surgical data dashboard can be adjusted based on a user selection.

In some aspects, the at least one machine learning model includes a post-operative machine learning model that analyzes the surgical data with respect to an output of a surgical machine learning model that analyzes the surgical data during a surgical procedure.

In some aspects, the one or more metrics can include time based metrics, camera position based metrics, efficiency based metrics, error based metrics, outlier based metrics, workflow based metrics, safety based metrics, variation based metrics, complexity metrics, predictive metrics, surgical tool movement based metrics, surgeon learning based metrics, and/or other metrics.

In some aspects, the surgical data dashboard can include a cases of interest interface, a recommended for you interface, and a change control that provides a selectable filter to control data selection in displaying the surgical data dashboard.

In some aspects, the surgical data dashboard can also include a workflow analysis view that compares a surgical workflow and other metrics associated with a surgical procedure, a robotic analysis view, a reachability metric view, a performance view, an error tracking view, an instrument analysis view, and a case summary view.

In some aspects, the robotic analysis view can include a filter control and an expand control that is user selectable to change a level of detail depicted through the robotic analysis view.

In some aspects, the reachability metric view can include a time base selector to set a time base for tracking data display and one or more recommendations identifying potential performance improvements.

In some aspects, the performance view can include a learning curve selector to group data for proficiency ratings and one or more recommendations identifying potential performance improvements, the error tracking view can include a tracking control to select a data source for tracking plotting and a division control to define how data is broken down for summarizing and display, and the instrument analysis view comprises an instrument standardization and instrument usage with separate time base controls.

In some aspects, a computer program product includes a memory device with computer-readable instructions stored thereon, where executing the computer-readable instructions by one or more processing units causes the one or more processing units to perform a plurality of operations. The operations can include analyzing a surgical dataset including information associated with a plurality of surgical procedures performed, where the surgical dataset includes surgical tool data, surgical video data, and configuration information. The operations can also include determining one or more metrics that quantify a plurality of aspects of the surgical procedures and generating a surgical data dashboard that summarizes the aspects of the surgical procedures based on the one or more metrics including a case summary view that summarizes the surgical procedures as a plurality of cases and includes search interface to perform text-based searches to filter displayed content of the case summary view. The operations can further include adjusting one or more visualizations of the surgical data dashboard based on a user selection.

In some aspects, a case view can be displayed in the surgical data dashboard based on a case selection through the case summary view.

In some aspects, the case view can include a video interface to play video of a selected case, a timeline, and a rating interface.

In some aspects, the surgical data dashboard can include a cases of interest interface, a recommended for you interface, and a change control that provides a selectable filter to control data selection in displaying the surgical data dashboard.

Turning now to FIG. 13, a computer system 300 is generally shown in accordance with an aspect. The computer system 300 can be an electronic computer framework comprising and/or employing any number and combination of computing devices and networks utilizing various communication technologies, as described herein. The computer system 300 can be easily scalable, extensible, and modular, with the ability to change to different services or reconfigure some features independently of others. The computer system 300 may be, for example, a server, desktop computer, laptop computer, tablet computer, or smartphone. In some examples, computer system 300 may be a cloud computing node. Computer system 300 may be described in the general context of computer-executable instructions, such as program modules, being executed by a computer system. Generally, program modules may include routines, programs, objects, components, logic, data structures, and so on that perform particular tasks or implement particular abstract data types. Computer system 300 may be practiced in distributed cloud computing environments where tasks are performed by remote processing devices that are linked through a communications network. In a distributed cloud computing environment, program modules may be located in both local and remote computer system storage media, including memory storage devices.

As shown in FIG. 13, the computer system 300 has one or more central processing units (CPU(s)) 301a, 301b, 301c, etc. (collectively or generically referred to as processor(s) 301). The processors 301 can be a single-core processor, multi-core processor, computing cluster, or any number of other configurations. The processors 301 can be any type of circuitry capable of executing instructions. The processors 301, also referred to as processing circuits, are coupled via a system bus 302 to a system memory 303 and various other components. The system memory 303 can include one or more memory devices, such as read-only memory (ROM) 304 and a random-access memory (RAM) 305. The ROM 304 is coupled to the system bus 302 and may include a basic input/output system (BIOS), which controls certain basic functions of the computer system 300. The RAM is read-write memory coupled to the system bus 302 for use by the processors 301. The system memory 303 provides temporary memory space for operations of said instructions during operation. The system memory 303 can include random access memory (RAM), read-only memory, flash memory, or any other suitable memory systems.

The computer system 300 comprises an input/output (I/O) adapter 306 and a communications adapter 307 coupled to the system bus 302. The I/O adapter 306 may be a small computer system interface (SCSI) adapter that communicates with a hard disk 308 and/or any other similar component. The I/O adapter 306 and the hard disk 308 are collectively referred to herein as a mass storage 310.

Software 311 for execution on the computer system 300 may be stored in the mass storage 310. The mass storage 310 is an example of a tangible storage medium readable by the processors 301, where the software 311 is stored as instructions for execution by the processors 301 to cause the computer system 300 to operate, such as is described hereinbelow with respect to the various Figures. Examples of computer program product and the execution of such instruction is discussed herein in more detail. The communications adapter 307 interconnects the system bus 302 with a network 312, which may be an outside network, enabling the computer system 300 to communicate with other such systems. In one aspect, a portion of the system memory 303 and the mass storage 310 collectively store an operating system, which may be any appropriate operating system to coordinate the functions of the various components shown in FIG. 13.

Additional input/output devices are shown as connected to the system bus 302 via a display adapter 315 and an interface adapter 316. In one aspect, the adapters 306, 307, 315, and 316 may be connected to one or more I/O buses that are connected to the system bus 302 via an intermediate bus bridge (not shown). A display 319 (e.g., a screen or a display monitor) is connected to the system bus 302 by a display adapter 315, which may include a graphics controller to improve the performance of graphics-intensive applications and a video controller. A keyboard, a mouse, a touchscreen, one or more buttons, a speaker, etc., can be interconnected to the system bus 302 via the interface adapter 316, which may include, for example, a Super I/O chip integrating multiple device adapters into a single integrated circuit. Suitable I/O buses for connecting peripheral devices such as hard disk controllers, network adapters, and graphics adapters typically include common protocols, such as the Peripheral Component Interconnect (PCI). Thus, as configured in FIG. 13, the computer system 300 includes processing capability in the form of the processors 301, and storage capability including the system memory 303 and the mass storage 310, input means such as the buttons, touchscreen, and output capability including the speaker 323 and the display 319.

In some aspects, the communications adapter 307 can transmit data using any suitable interface or protocol, such as the internet small computer system interface, among others. The network 312 may be a cellular network, a radio network, a wide area network (WAN), a local area network (LAN), or the Internet, among others. An external computing device may connect to the computer system 300 through the network 312. In some examples, an external computing device may be an external web server or a cloud computing node.

It is to be understood that the block diagram of FIG. 13 is not intended to indicate that the computer system 300 is to include all of the components shown in FIG. 13. Rather, the computer system 300 can include any appropriate fewer or additional components not illustrated in FIG. 13 (e.g., additional memory components, embedded controllers, modules, additional network interfaces, etc.). Further, the aspects described herein with respect to computer system 300 may be implemented with any appropriate logic, wherein the logic, as referred to herein, can include any suitable hardware (e.g., a processor, an embedded controller, or an application-specific integrated circuit, among others), software (e.g., an application, among others), firmware, or any suitable combination of hardware, software, and firmware, in various aspects. Various aspects can be combined to include two or more of the aspects described herein.

Aspects disclosed herein may be a system, a method, and/or a computer program product at any possible technical detail level of integration. The computer program product may include a computer-readable storage medium (or media) having computer-readable program instructions thereon for causing a processor to carry out various aspects.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present disclosure may be assembler instructions, instruction-set-architecture (ISA) instructions, machine instructions, machine-dependent instructions, microcode, firmware instructions, state-setting data, configuration data for integrated circuitry, or either source-code or object code written in any combination of one or more programming languages, including an object-oriented programming language, such as Smalltalk, C++, high-level languages such as Python, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some aspects, electronic circuitry including, for example, programmable logic circuitry, field-programmable gate arrays (FPGA), or programmable logic arrays (PLA) may execute the computer-readable program instruction by utilizing state information of the computer-readable program instructions to personalize the electronic circuitry, in order to perform aspects of the present disclosure.

Aspects are described herein with reference to flowchart illustrations and/or block diagrams of methods, apparatus (systems), and computer program products according to aspects of the disclosure. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a computer system, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other devices to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various aspects. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various aspects have been presented for purposes of illustration but are not intended to be exhaustive or limited to the aspects disclosed. Many modifications and variations will be apparent to those of ordinary skill in the art without departing from the scope and spirit of the described aspects. The terminology used herein was chosen to best explain the principles of the aspects, the practical application or technical improvement over technologies found in the marketplace, or to enable others of ordinary skill in the art to understand the aspects described herein.

Various aspects are described herein with reference to the related drawings. Alternative aspects can be devised without departing from the scope of this disclosure. Various connections and positional relationships (e.g., over, below, adjacent, etc.) are set forth between elements in the following description and in the drawings. These connections and/or positional relationships, unless specified otherwise, can be direct or indirect, and the present disclosure is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains,” or “containing,” or any other variation thereof are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any aspect or design described herein as “exemplary” is not necessarily to be construed as preferred or advantageous over other aspects or designs. The terms “at least one” and “one or more” may be understood to include any integer number greater than or equal to one, i.e., one, two, three, four, etc. The terms “a plurality” may be understood to include any integer number greater than or equal to two, i.e., two, three, four, five, etc. The term “connection” may include both an indirect “connection” and a direct “connection.”

The terms “about,” “substantially,” “approximately,” and variations thereof are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium, such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), graphics processing units (GPUs), microprocessors, application-specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A computer-implemented method comprising:
analyzing, by a processor, a surgical dataset comprising information associated with a plurality of surgical procedures performed;
determining, by the processor, one or more metrics that quantify a plurality of aspects of the surgical procedures;
generating, by the processor, a surgical data dashboard that summarizes the aspects of the surgical procedures based on the one or more metrics including a workflow analysis with a filter control; and
adjusting, by the processor, one or more visualizations of the surgical data dashboard based on a user selection through the filter control.

2. The computer-implemented method of claim 1, wherein the one or more metrics are displayed in user interactive elements.

3. The computer-implemented method of claim 1 or claim 2, wherein the surgical dataset comprises two or more of: surgical tool data, surgical video data, and/or configuration information.

4. The computer-implemented method of any preceding claim, wherein the one or more metrics comprise time based metrics, camera position based metrics, efficiency based metrics, error based metrics, outlier based metrics, workflow based metrics, and/or safety based metrics.

5. The computer-implemented method of any one of claims 1 to 3, wherein the one or more metrics comprise variation based metrics, complexity metrics, predictive metrics, surgical tool movement based metrics, and/or surgeon learning based metrics.

6. The computer-implemented method of any preceding claim, wherein the surgical data dashboard comprises a cases of interest interface, a recommended for you interface, and a change control that provides a selectable filter to control data selection in displaying the surgical data dashboard.

7. The computer-implemented method of any preceding claim, wherein the surgical data dashboard comprises one or more of: a workflow analysis view that compares a surgical workflow and other metrics associated with a surgical procedure, a workflow variation view, a robotic analysis view, a reachability metric view, a performance view, an error tracking view, an instrument analysis view, and a case summary view.

8. A system comprising:
a memory system; and
a processing system coupled to the memory system and configured to execute instructions to perform a plurality of operations comprising:
analyzing a surgical dataset comprising information associated with a plurality of surgical procedures performed, wherein the surgical dataset comprises two or more of: surgical tool data, surgical video data, and/or configuration information;
determining one or more metrics that quantify a plurality of aspects of the surgical procedures, wherein at least one of the one or more metrics is determined based on at least one machine learning model;
generating a surgical data dashboard that summarizes the aspects of the surgical procedures based on the one or more metrics including a workflow variation view configured to display a workflow standardization and a variation over surgery; and
adjusting one or more visualizations of the surgical data dashboard based on a user selection.

9. The system of claim 8, wherein the at least one machine learning model comprises a post-operative machine learning model that analyzes the surgical data with respect to an output of a surgical machine learning model that analyzes the surgical data during a surgical procedure.

10. The system of claim 8 or claim 9, wherein the one or more metrics comprise time based metrics, camera position based metrics, efficiency based metrics, error based metrics, outlier based metrics, workflow based metrics, safety based metrics, variation based metrics, complexity metrics, predictive metrics, surgical tool movement based metrics, and/or surgeon learning based metrics.

11. The system of any one of claims 8 to 10, wherein the surgical data dashboard comprises a cases of interest interface, a recommended for you interface, and a change control that provides a selectable filter to control data selection in displaying the surgical data dashboard.

12. The system of any one of claims 8 to 11, wherein the surgical data dashboard comprises a workflow analysis view that compares a surgical workflow and other metrics associated with a surgical procedure, a robotic analysis view, a reachability metric view, a performance view, an error tracking view, an instrument analysis view, and a case summary view.

13. The system of claim 12, wherein the robotic analysis view comprises a filter control and an expand control that is user selectable to change a level of detail depicted through the robotic analysis view.

14. The system of claim 12, wherein the reachability metric view comprises a time base selector to set a time base for tracking data display and one or more recommendations identifying potential performance improvements.

15. The system of claim 12, wherein the performance view comprises a learning curve selector to group data for proficiency ratings and one or more recommendations identifying potential performance improvements, the error tracking view comprises a tracking control to select a data source for tracking plotting and a division control to define how data is broken down for summarizing and display, and the instrument analysis view comprises an instrument standardization and instrument usage with separate time base controls.

16. A computer program product comprising a memory device with computer-readable instructions stored thereon, wherein executing the computer-readable instructions by one or more processing units causes the one or more processing units to perform a plurality of operations comprising:
analyzing a surgical dataset comprising information associated with a plurality of surgical procedures performed, wherein the surgical dataset comprises surgical tool data, surgical video data, and configuration information;
determining one or more metrics that quantify a plurality of aspects of the surgical procedures;
generating a surgical data dashboard that summarizes the aspects of the surgical procedures based on the one or more metrics including a case summary view that summarizes the surgical procedures as a plurality of cases and includes search interface to perform text-based searches to filter displayed content of the case summary view; and
adjusting one or more visualizations of the surgical data dashboard based on a user selection.

17. The computer program product of claim 16, wherein a case view is displayed in the surgical data dashboard based on a case selection through the case summary view.

18. The computer program product of claim 17, wherein the case view comprises a video interface to play video of a selected case, a timeline, and a rating interface.

19. The computer program product of any one of claims 16 to 18, wherein the one or more metrics comprise time based metrics, camera position based metrics, efficiency based metrics, error based metrics, outlier based metrics, workflow based metrics, safety based metrics, variation based metrics, complexity metrics, predictive metrics, surgical tool movement based metrics, and/or surgeon learning based metrics.

20. The computer program product of any one of claims 16 to 19, wherein the surgical data dashboard comprises a cases of interest interface, a recommended for you interface, and a change control that provides a selectable filter to control data selection in displaying the surgical data dashboard.
